(19) United States
(12) Patent Application Publication
(10) Pub. No.: US 2016/0070857 A1
(43) Pub. Date: Mar. 10, 2016

(54) METHOD FOR EXTRACTING BIOMARKER FOR DIAGNOSING BILIARY TRACT CANCER, COMPUTING APPARATUS THEREFOR, BIOMARKER FOR DIAGNOSING BILIARY TRACT CANCER AND APPARATUS FOR DIAGNOSING BILIARY TRACT CANCER COMPRISING SAME

(71) Applicants: LG ELECTRONICS INC., Seoul (KR); INDUSTRY-ACADEMIC COOPERATION FOUNDATION, YONSEI UNIVERSITY, Seoul (KR)

(72) Inventors: Hyungseok CHOI, Seoul (KR); Jeeyeon HEO, Seoul (KR); Siyoung SONG, Seoul (KR); Dawoon JUNG, Seoul (KR)

(73) Assignees: LG ELECTRONICS INC., Seoul (KR); INDUSTRY-ACADEMIC COOPERATION FOUNDATION, YONSEI UNIVERSITY, Seoul (KR)

(21) Appl. No.: 14/785,166

(22) PCT Filed: Apr. 16, 2014
(86) PCT No.: PCT/KR2014/003299

§ 371 Date: Oct. 16, 2015

(30) Foreign Application Priority Data
Apr. 17, 2013 (KR) 10-2013-0042328
Oct. 15, 2013 (KR) 10-2013-0122635

(51) Int. Cl. G06F 19/24; C12Q 1/68

(52) U.S. Cl. CPC G06F 19/24; C12Q 1/6886; C12Q 2600/178

(57) ABSTRACT

Disclosed are a method for extracting a biomarker for diagnosing biliary tract cancer and a computing apparatus therefor, and the biomarker for diagnosing biliary tract cancer and an apparatus for diagnosing biliary tract cancer comprising same. More particularly, disclosed are a method for extracting a biomarker for diagnosing biliary tract cancer using a gene specifically expressed in a biliary tract cancer patient, or a microRNA obtained from blood or a tissue, capable of forming a pair with the gene and a computing apparatus therefor, and the biomarker for diagnosing biliary tract cancer and an apparatus for diagnosing biliary tract cancer comprising same.

### TECHNICAL FIELD

The present invention relates to a method for extracting a biomarker for diagnosing biliary tract cancer and a computing apparatus therefor, and the biomarker for diagnosing biliary tract cancer and an apparatus for diagnosing biliary tract cancer comprising the same. More specifically, the present invention relates to a method for extracting a biomarker for diagnosing biliary tract cancer by using a microRNA obtained from blood or a tissue and a computing apparatus therefor, and the biomarker for diagnosing biliary tract cancer and an apparatus for diagnosing biliary tract cancer comprising the same.

### BACKGROUND ART

Bile ducts serve to send bile made by a liver to a duodenum. The bile ducts become thicker while they are gradually joined with one another like that twigs are collected toward one bough in a liver, and both bile ducts are joined with one when coming out of the liver. The bile ducts are classified into an intrahepatic bile duct passing through the inside of the liver and an extrahepatic bile duct connected to the duodenum by coming off the liver. In the extrahepatic bile duct, a portion for temporarily storing and concentrating bile is referred to as gall bladder, and the bile ducts and the gall bladder inside and outside the liver are referred to as biliary tract.

Biliary tract cancer is also referred to as bile duct cancer, and is a malignant tumor generated from the epithelium of the bile duct. The biliary tract cancer may be classified into two types, that is, intrahepatic biliary tract cancer and extrahepatic biliary tract cancer depending on a site where a malignant tumor is generated. Generally, the biliary tract cancer mainly indicates a cancer generated in an extrahepatic bile duct. In this specification, the biliary tract cancer will refer to both intrahepatic biliary tract cancer and extrahepatic biliary tract cancer unless specified otherwise.

It is not easy to exactly identify and diagnose a tumor mass of the biliary tract cancer because the biliary tract cancer is likely to be spread by being permeated into peripheral tissues and does not form an explicit tumor mass. Recently, with the development of image diagnosis techniques, the biliary tract cancer has been diagnosed using techniques of abdominal ultrasonography, computed tomography (CT), magnetic resonance image (MRI), Percutaneous Transhepatic Cholangiography (PTC), percutaneous transhepatic biliary drainage (PTBD), endoscopic retrograde cholangiopancreatography (ERCP) or angiography. However, such image diagnosis techniques cause high cost in diagnosis and are complicated, and are no good for early diagnosis in fact, whereby the development of a biomarker for early diagnosis of biliary tract cancer will be required necessarily.

In this respect, although several tens of biomarkers for other cancer species have been developed for the last 20 years, biomarkers for biliary tract cancer have not been commercialized until now.

Meanwhile, micro RNA (miRNA) means a short single strand non-coding RNA molecule comprised of 17 to 25 nucleotides, approximately. It is known that the micro RNA controls proteinogenous gene expression by disturbing a transcription process of target mRNA (gene) or decomposing mRNA. It is also known that the micro RNA exists in blood as well as tissue.

Also, the development of a biomarker based on tissue or blood samples will be required for easiness of handling and diagnosis. Particularly, blood samples will be useful.

### DISCLOSURE

### Technical Problem

To solve the aforementioned problems, an object of the present invention is to provide a method for extracting a biomarker for diagnosing biliary tract cancer including biliary tract cancer patient specific gene combination or a method for extracting a biomarker for diagnosing biliary tract cancer by using micro RNA obtained from blood or tissue and a computing apparatus therefor. Also, the present invention is to provide a biomarker for diagnosing biliary tract cancer and an apparatus for diagnosing biliary tract cancer comprising the same.

It will be appreciated by persons skilled in the art that the objects that could be achieved with the present invention are not limited to what has been particularly described hereinabove and the above and other objects that the present invention could achieve will be more clearly understood from the following detailed description.

### Technical Solution

A method for extracting a biomarker for diagnosing biliary tract cancer according to one embodiment of the present invention comprises the steps of computing interaction scores obtained by digitalizing complementary combination levels between microRNAs and genes; determining n number of microRNA and gene pairs having high interaction scores; and extracting either a gene common to a gene specifically expressed from biliary tract cancer patients among the n number of microRNA and gene pairs or microRNA which is the pair of the gene.

A biomarker for diagnosing biliary tract cancer according to one embodiment of the present invention comprises ACSM5, ADH6, ALDH1L1, APOA5, BHMT, CCL16, CYP1A2, CYP3A43, DAO, DDC, ESR1, F11, F13B, FETUB, GLYAT, GNMT, IGFALS, NAT2, PFKFB1, RDH16, SRD5A2, SULT2A1 and THRSP.

In another aspect, a biomarker for diagnosing biliary tract cancer according to one embodiment of the present invention comprises hsa-miR-21-5p, hsa-miR-93-5p, hsa-miR-106b-5p, hsa-miR-155-5p, hsa-miR-181a-5p, hsa-miR-200a-3p, hsa-miR-200b-3p, hsa-miR-222-3p, and hsa-miR-331-3p, which use tissue as a biological sample.

In other aspect, a biomarker for diagnosing biliary tract cancer according to one embodiment of the present invention comprises hsa-miR-21-5p, hsa-miR-93-5p, hsa-miR-106b-5p, hsa-miR-155-5p, hsa-miR-181a-5p, and hsa-miR-222-3p, which use blood as a biological sample.

An apparatus for diagnosing biliary tract cancer according to one embodiment of the present invention includes any one of the aforementioned biomarkers.

It will be appreciated by persons skilled in the art that the objects that could be achieved with the present invention are not limited to what has been particularly described hereinabove and the above and other solutions that the present invention could achieve will be more clearly understood from the following detailed description.

### Advantageous Effects

The present invention may provide a method for extracting a biomarker for diagnosing biliary tract cancer. The present invention may provide a biomarker for diagnosing biliary tract cancer, which has high specificity and sensitivity. Also, the present invention may provide an apparatus for diagnosing biliary tract cancer, which includes the aforementioned biomarker.

It will be appreciated by persons skilled in the art that that the effects that can be achieved through the present invention are not limited to what has been particularly described hereinabove and other advantages of the present invention will be more clearly understood from the following detailed description.

### BRIEF DESCRIPTION OF THE DRAWINGS

The accompanying drawings, which are included to provide a further understanding of the invention, illustrate embodiments of the invention and together with the description serve to explain the principle of the invention. In the drawings:

FIG. 1 is a block diagram illustrating a computing apparatus according to the present invention;

FIG. 2 is a conceptual diagram illustrating an example of computing of interaction scores between miRNAs and genes;

FIG. 3 is a flow chart illustrating a computing method of interaction scores;

FIG. 4 is a conceptual diagram illustrating a method for computing a correlation value between a similar miRNA and a specific gene by using a similarity database;

FIG. 5 is a flow chart illustrating a method for computing a correlation value between a similar miRNA and a gene by using a similarity database;

FIG. 6 is a conceptual diagram illustrating a method for computing a correlation value between a neighboring miRNA and a specific gene by using a miRNA cluster database;

FIG. 7 is a flow chart illustrating a method for computing a weight value between a neighboring miRNA and a specific gene by using a miRNA cluster database;

FIG. 8 is a conceptual diagram illustrating a method for computing a correlation value between a specific miRNA and a transcription control gene by using a transcription factor database;

FIG. 9 is a flow chart illustrating a method for computing a weight value between a specific miRNA and a transcription control gene by using a transcription factor database;

FIG. 10 is a flow chart illustrating a method for extracting a biomarker for a biliary tract cancer patient on the basis of an integrated analysis algorithm for extracting the biomarker;

FIG. 11 illustrates a plot of an analysis result of principal components based on data GSE26566;

FIG. 12 illustrates a heat map of a hierarchical cluster analysis result based on data GSE26566;

FIG. 13 illustrates a heat map of a hierarchical cluster analysis result based on data GSE32957;

FIG. 14 is a conceptual diagram illustrating a small RNA sequencing data analysis which is one of detailed examples of next generation genome sequencing;

FIG. 15 illustrates a hierarchical cluster analysis result based on next generation genome sequencing data indicating expression patterns of 9 micro RNAs expressed from a tissue sample in accordance with the present invention; and

FIG. 16 illustrates a hierarchical cluster analysis result based on next generation genome sequencing data indicating expression patterns of 6 micro RNAs expressed from a blood sample in accordance with the present invention.

### BEST MODE FOR CARRYING OUT THE INVENTION

Hereinafter, a computing apparatus related to the present invention will be described in more detail with reference to the accompanying drawings.

A suffix such as “module” and “unit” used in the following description is merely intended to facilitate description of the specification, and the suffix itself is not intended to give any special meaning or function.

The present invention discloses a computing apparatus 100 to which an integrated analysis algorithm for extracting a biomarker is applied, and the biomarker extracted through the computing apparatus 100. In this case, the computing apparatus 100 described herein may include a high speed computing apparatus based on an electronic circuit such as a personal computer, a workstation, and a super computer. In addition to a fixed apparatus such as a computer, a workstation and a super computer, a mobile apparatus such as a smart phone, a PDA, and a laptop computer, which includes a central processing unit and may perform computation processing, may be included in the computing apparatus.

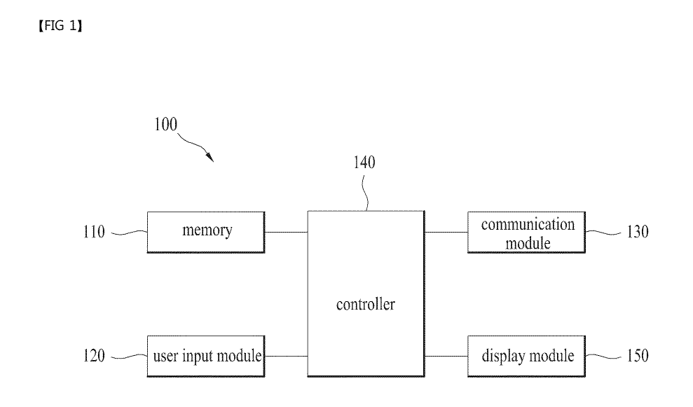

FIG. 1 is a block diagram illustrating a computing apparatus according to the present invention. Referring to FIG. 1, the computing apparatus 100 according to the present invention may include a memory 110, a user input module 120, a communication module 130, and a controller 140.

The memory 110 may store a program for an operation of the controller 140, and may temporarily store input and output data (for example, database). Moreover, the memory 110 may store data transmitted and received by the communication module 130 during communication.

The memory 110 may include at least one of a flash memory type, a hard disk type, a multimedia card micro type, a card type memory (for example, SD or XD memory 110), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk.

The user input module 120 serves to receive a user input from a user. The user input module 120 may include a key board, a mouse, etc.

The communication module 130 serves to receive data from the outside or transmit data to the outside through communication. The communication module 130 according to the present invention may serve to receive various kinds of databases from a remote server.

The controller 140 controls an overall operation of the computing apparatus 100, and performs various kinds of computations. The controller 140 according to the present invention may compute interaction scores and correlation values, which will be described later, and may perform computation for extracting a biomarker for diagnosing biliary tract cancer.

The computing apparatus 100 according to the present invention may further include a display module 150 for outputting information. The display module 150 may display an input of a user and serve as an output device for outputting the result of computation of the controller 140. The display module 150 may be a device such as a monitor for assisting the computing apparatus 100.

The embodiments which will be described below are not limited to the computing apparatus 100 described as above, and the computing apparatus 100 may be configured by selective combination of all or some of the following embodiments such that various modifications may be made in the following embodiments.

A method for extracting a biomarker for diagnosing biliary tract cancer will be described in detail by using the aforementioned computing apparatus 100.

An integrated analysis algorithm for extracting a biomarker, which is described in the present invention, may be configured by combination of a differentially expressed genes analysis algorithm and a micro RNA target genes analysis algorithm.

First of all, the differentially expressed genes analysis algorithm will be described. The differentially expressed genes analysis algorithm is an algorithm for statistically meaningfully discovering genes, which are over-expressed or low-expressed, differently from normal people, from biliary tract cancer patients, and is intended to discover genes that may identify a normal people group from a patient group by using a linear model which is one of advanced statistical methods (reference document, Statistical Applications in Genetics and Molecular Biology, Vol. 3, No. 1, Article 3).

The differentially expressed genes analysis algorithm may be categorized into a data normalization step and a statistical analysis step. The data normalization step is to integrate and correct microarray data for all human genes obtained from a normal people group and a patient group. For data normalization, a robust multichip average (RMA) algorithm may be used (reference document, Biostatistics, Vol. 4, No. 2, 249-264).

The statistical analysis step is to select genes having a statistically meaningful difference in the amount of expression between two groups (that is, normal people group and patient group) by using a linear model of normalized data. The statistical meaningful probability may select genes of which q-value is 0.01 or less, which is a p-value corrected using a FDR (False Discovery Rate) method (reference document, Journal of the Royal Statistical Society, Series B (Methodological), Vol. 57, No. 1, 289-300).

The computing apparatus 100 of the present invention may use a list of genes specific-expressed (over-expressed or low-expressed) from biliary tract cancer patients by using the differentially expressed genes analysis algorithm to extract a biomarker for diagnosing biliary tract cancer. Such a discovery of the list of genes specific-expressed from biliary tract cancer patients by using the differentially expressed genes analysis algorithm is a technique which is already known, and thus its detailed description will be omitted.

Next, the micro RNA target genes analysis algorithm will be described. The micro RNA target genes analysis algorithm described in the present invention is to provide a statistical equation that exactly discovers micro RNA target genes by using at least one of a micro RNA target genes prediction calculated value obtained from the conventional miro RNA database, a correlation calculated value between expression patterns of micro RNAs and genes obtained through a microarray experiment, and a weight value calculated value based on a biological mechanism.

Hereinafter, a computing method of the micro RNA target genes prediction calculated value (or interaction scores), the correlation calculated value and the weight calculated value will be described in detail. For convenience of description, it is assumed that miRNA and gene disclosed in the present invention will be equivalent to micro RNA and genes, respectively.

Computation of Micro RNA Target Genes Prediction Value

The computing apparatus 100 according to the present invention may compute interaction scores obtained by digitalizing complementary combination levels between micro RNA and its target genes. A high possibility or low possibility of complementary combination between micro RNA and its target genes may be determined through the interaction scores. A computing method of the interaction scores will be described in detail with reference to the drawings which will be described later.

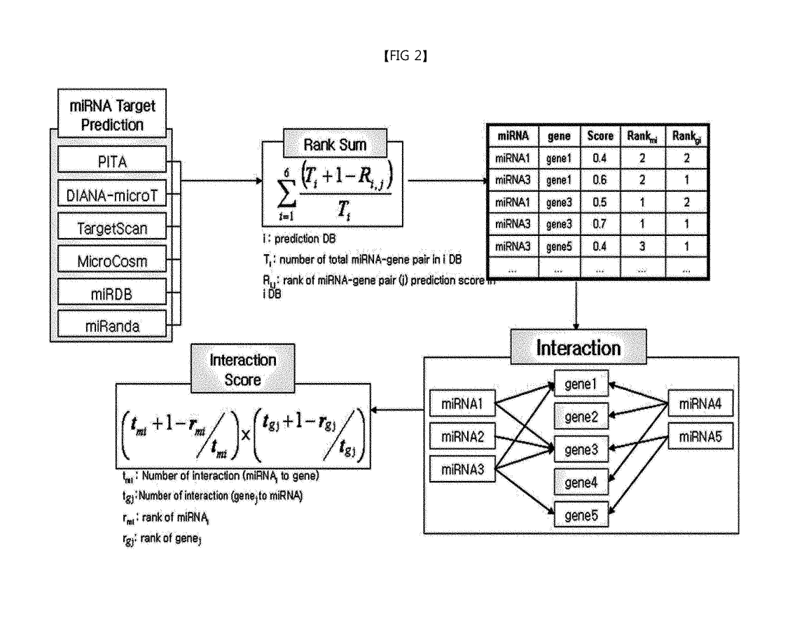

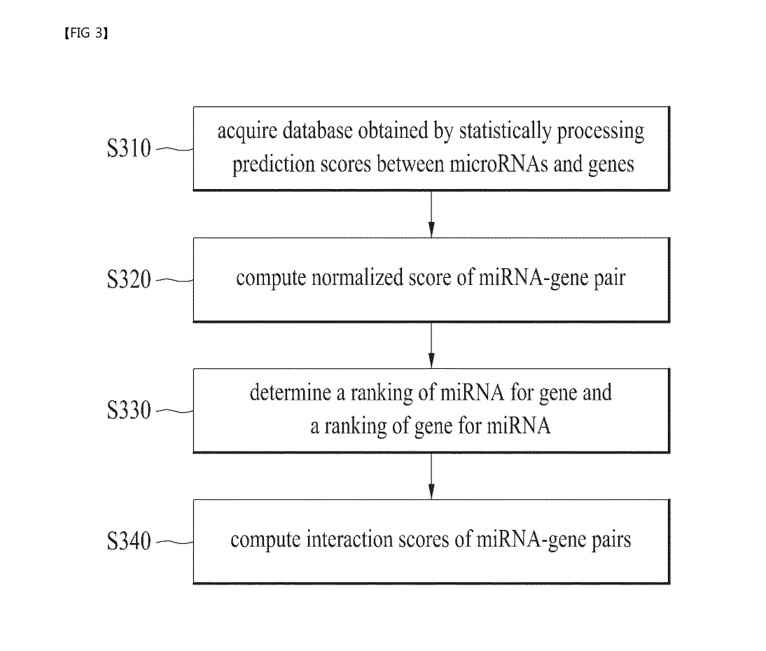

FIG. 2 is a conceptual diagram illustrating an example of computing of interaction scores between miRNAs and genes, and FIG. 3 is a flow chart illustrating a computing method of interaction scores.

Referring to FIGS. 2 and 3, first of all, the computing apparatus 100 may acquire a statistically processed database of prediction scores between miRNA and genes by using at least one or more miRNA target prediction tools (S310).

The miRNA target prediction tool may mean a software tool obtained by digitalizing combination levels of a target gene and miRNA pair, which may be complementarily combined with the target gene to inhibit a process of making the target gene be a protein. Examples of the miRNA target prediction tool for acquiring prediction scores of gene-miRNA pairs may include Targetscan, miRDB, DIANA-microT, PITA, miRanda MicroCosm, RNAhybrid, PicTar, RNA22, etc. A brief description of each of the miRNA target prediction tools is made in Table 1.

TABLE 1

| Tool name | Tool description (use information) | Reference site |
|---|---|---|
| Targetscan | sequence similarity information and | http://www.ncbi.nlm.nih.gov/pubmed/18955434 |
| | conservation information | |
| miRDB | sequence similarity information, | http://www.ncbi.nlm.nih.gov/pubmed/18426918 |
| | thermodynamic stability information, | |
| | and conservation information | |
| DIANA-microT | sequence similarity information and | http://www.ncbi.nlm.nih.gov/pubmed/15131085 |
| | thermodynamic stability information | |
| PITA | sequence similarity information and | http://www.ncbi.nlm.nih.gov/pubmed/17893677 |
| | thermodynamic stability information | |
| miRanda | thermodynamic stability information | http://www.ncbi.nlm.nih.gov/pubmed/14709173 |
| | and conservation information | |
| MicroCosm | thermodynamic stability information | http://www.ebi.ac.uk/enright-srv/microcosm/htdocs/targets/v5/info.html |
| | and conservation information | |
| RNAhybrid | thermodynamic stability information | http://www.ncbi.nlm.nih.gov/pubmed/15383676 |
| PicTar | thermodynamic stability information | http://www.ncbi.nlm.nih.gov/pubmed/15806104 |
| | and conservation information | |
| RNA22 | sequence pattern information | http://www.ncbi.nlm.nih.gov/pubmed/16990141 |

If the target prediction tool is used, prediction scores between various genes that may complementarily be combined with miRNA may be scored. If the prediction scores are small, it may mean that the possibility of complementary combination between miRNA and gene is lowered.

The target prediction tool may be driven by the computing apparatus 100 according to the present invention, and a statistically processed database of prediction scores of miRNA-gene pairs may be acquired by, but not limited to, computation of the controller 140. The computing apparatus 100 according to the present invention may acquire the statistically processed database of the prediction scores of miRNA-gene pairs from a remote server which uses the target prediction tool.

In order to increase reliability of the prediction scores between the miRNA-gene pairs, it is preferable to acquire a plurality of databases by using a plurality of target prediction tools rather than using one target prediction tool. In FIG. 2, PITA, DIANA-microT, TargetScan, MicroCosm, miRDB and miRanda are used as the target prediction tools.

If the statistically processed database of the prediction scores of miRNA-gene pairs are acquired using the plurality of target prediction tools, the controller 140 may compute normalized scores on the basis of a ranking of the prediction scores of the miRNA-gene pairs to normalize the database (S320).

As shown in the example of Table 1, information used by the miRNA target prediction tool may be varied and different units may be applied to calculation of the prediction score for each database. Therefore, if the plurality of databases are used, normalization of the databases may be required necessarily. In order to normalize the prediction scores of the miRNA-gene pairs, the controller 140 may determine the ranking based on the prediction scores of the miRNA-gene pairs for each database, convert the prediction scores to standard scores, and acquire the normalized scores by adding up the standard scores of the miRNA-gene pairs in each data base. The Equation 1 illustrates that it is used to acquire the normalized scores.

$${\sum }_{i = 1}^{n} \frac{( {T}_{i} + 1 - {R}_{i , j} )}{{T}_{i}} \qquad [ Equation 1 ]$$

In the Equation 1, i may mean the ith database, n may mean the number of databases (for example, since six databases may be acquired through six prediction tools in FIG. 2, n may be set to 6), Ti may mean a total number of miRNA-gene pairs in the ith database, and Ri,j may mean the ranking of the jth miRNA-gene pairs in the ith database.

For example, if a prediction score of a miRNA1-gene1 pair corresponds to the ranking 20 of 100 pairs in the first database where 100 miRNA-gene pairs exist, the standard score of the miRNA-gene1 in the first database will be (100+1-20)/100=0.81. The controller 140 may compute the normalized score of the miRNA1-gene1 pair by adding up the standard scores of the miRNA1-gene1 pair in the second to nth databases.

Afterwards, the controller 140 may determine the ranking of miRNAs for a specific gene and the ranking of genes for a specific miRNA on the basis of the normalized score (S330).

For example, when miRNAs which may be complementarily combined with gene1 are miRNA1, miRNA3, and miRNA4, the controller 140 may determine the ranking of miRNA in the order of higher complementary combination with gene1 (that is, in the order of higher normalized score) on the basis of each normalized score of gene1-miRNA1, gene1-miRNA3 and gene1-miRNA4. In FIG. 2, a normalized score between miRNA1-gene1 is set to 0.4, and a normalized score between miRNA3-gene1 is set to 0.6, whereby miRNA1 has the second ranking for gene1, and miRNA3 has the first ranking for gene1.

In this way, the ranking of genes for a specific miRNA may be determined. For example, if genes that may complementarily be combined with miRNA1 are gene1 and gene3, the controller 140 may determine the ranking of genes in the order of higher complementary combination with miRNA1 (that is, in the order of higher normalized score) on the basis of each normalized score of miRNA1-gene1 and miRNA1-gene3. In FIG. 2, a normalized score between miRNA1-gene1 is set to 0.4, and a normalized score between miRNA1-gene3 is set to 0.5, whereby gene1 has the second ranking for miRNA1, and gene3 has the first ranking for miRNA1.

Afterwards, the controller 140 may compute an interaction score between gene-miRNA on the basis of the ranking of genes and miRNAs (S340). The Equation 2 illustrates that it is used to compute the interaction score.

$$( \frac{{t}_{m i} + 1 - {r}_{m i}}{{t}_{m i}} ) \times ( \frac{{t}_{gj} + 1 - {r}_{gj}}{{t}_{gj}} ) \qquad [ Equation 2 ]$$

In the Equation 2, tmi may mean the number of ith miRNAi-gene pairs, tgj may mean the number of genej-miRNA pairs, rmi may mean the ranking of the normalized score of the ith miRNA for the jth gene, and rgj may mean the ranking of the normalized score of the jth gene for the ith miRNA.

Correlation Computation

The aforementioned target miRNA prediction tool has no database related to all miRNAs and all genes of a human body. In the present invention, interaction scores of various miRNAs and genes, which cannot be predicted from the target miRNA prediction tool, may be acquired by using similarity between miRNAs, a peripheral influence between miRNAs, and a transcription factor of genes.

### Embodiment 1

### Weight Computation Caused by Correlation

The computing apparatus 100 according to the present invention may acquire a correlation value between expression patterns of a specific miRNA and a specific gene, which are obtained through a microarray experiment, and may predict a correlation value between a similar miRNA similar to the specific miRNA and the specific gene. Computation of the correlation value between the similar miRNA and the specific gene will be described in detail with reference to the drawings which are disclosed later.

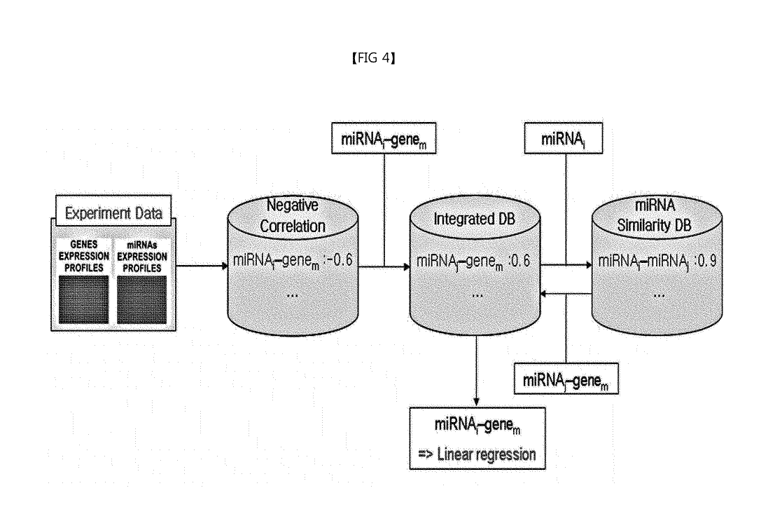

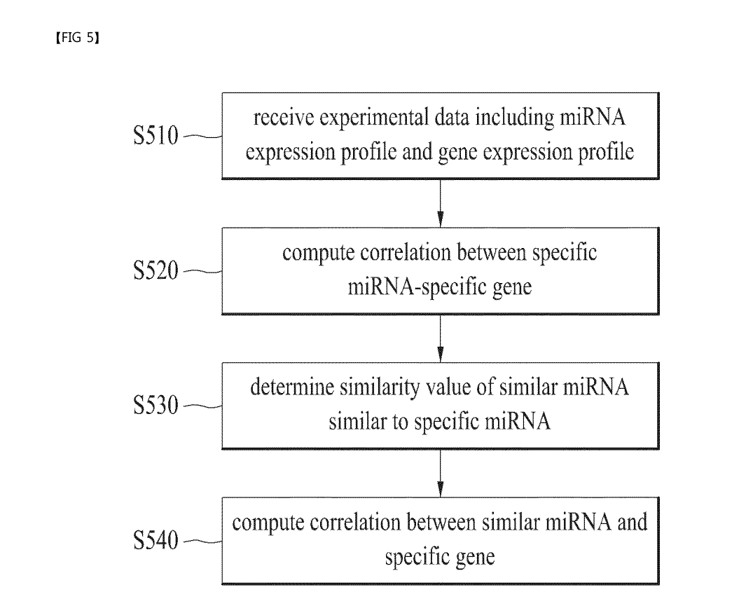

FIG. 4 is a conceptual diagram illustrating a method for computing a correlation value between a similar miRNA and a specific gene by using a similarity database, and FIG. 5 is a flow chart illustrating a method for computing a correlation value between a similar miRNA and a gene by using a similarity database.

First of all, if experimental data that include genes expression profiles and miRNA expression profiles, which are obtained through a microarray experiment, are input (S510), the controller 140 may compute the correlation between the specific miRNA and the specific gene on the basis of the input experimental data (S520).

The microarray experiment will be described preferentially. A gene microarray is a tool that may measure the amount of gene expression for all or some of genes of a living thing, and is also referred to as a DNA microarray. An observation capability of genes is extended to all of living things at an individual gene level due to the introduction of the gene, whereby the living things may be studied as one system. Also, since the gene microarray is performed at a large scale by basically parallelizing the conventional gene detection scheme, a remarkable change has been made even in a method for processing and analyzing data. In the method for performing a gene microarray, after several thousands of gene sequences to several ten thousands of gene sequences are fixed on a slide surface of 1 cm², RNA of a cell gathered from various experimental conditions is extracted and reverse-transcribed to be labeled with a fluorescent material. Subsequently, the labelled DNA is subjected to hybridization to the microarray and the hybridized DNA is scanned to form an image. Then, coloration strength based on a fluorescent material is measured per gene location by an image analysis program, whereby expression of genes and expression level are analyzed in comparison with quantified gene expression numerical data by using informatics such as mathematics, statistics and computer science.

An expression level between a specific miRNA and a specific gene may be digitalized through the above microarray experiment. The correlation between the specific miRNA and the specific gene is Pearson's correlation, and may indicate relative quantity of increase and reduction of the expression amount of the specific miRNA based on increase of the expression amount of the specific gene.

Afterwards, the computing apparatus 100 may acquire a similarity value of a similarity miRNA similar to the specific miRNA by using a miRNA similarity database (S530). The miRNA similarity database may include a similarity value obtained by digitalizing functional similarity between miRNAs. The miRNA similarity database may be acquired through a BLAST or BLAT tool, which is already known.

Afterwards, the computing apparatus 100 may compute the correlation between the similar miRNA and the specific gene by using the similarity value (S540). A linear regression model based on the similarity value may be used to compute a weight value between the similar miRNA and the gene.

### Embodiment 2

### Computation of Correlation in Consideration of miRNA Peripheral Influence

The computing apparatus 100 according to the present invention may compute a correlation value between a specific miRNA and a neighboring miRNA forming a cluster. Computation of the correlation value in consideration of an influence between miRNAs will be described with reference to the drawings which will be disclosed later.

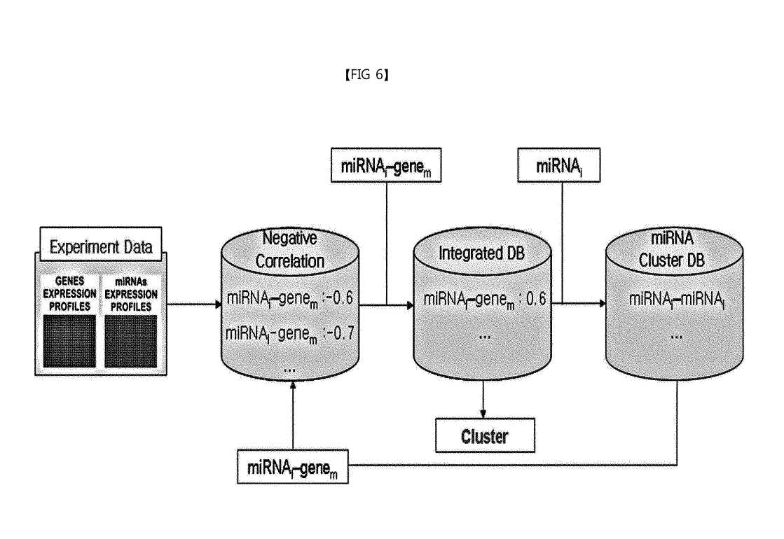

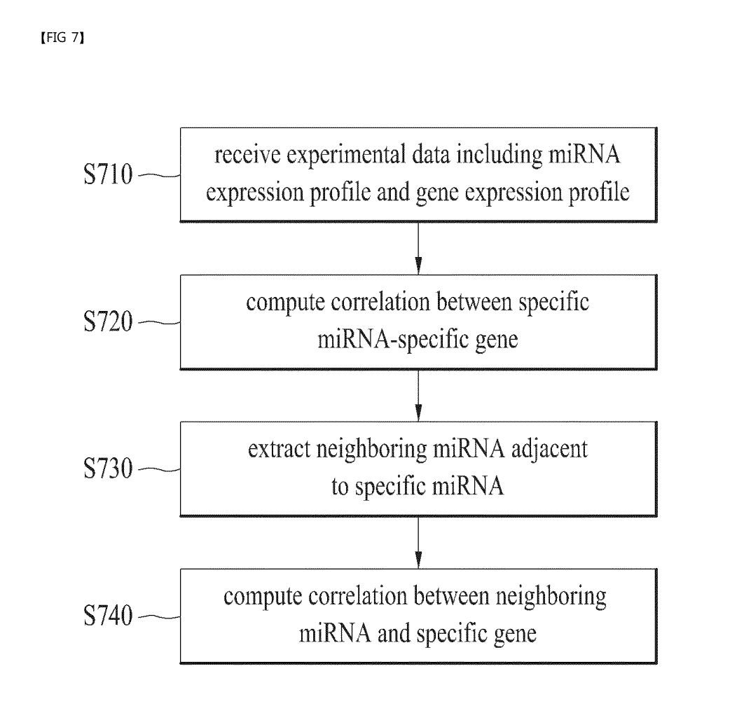

FIG. 6 is a conceptual diagram illustrating a method for computing a correlation value between a neighboring miRNA and a specific gene by using a miRNA cluster database, and FIG. 7 is a flow chart illustrating a method for computing a weight value between a neighboring miRNA and a specific gene by using a miRNA cluster database.

First of all, if experimental data that include genes expression profiles and miRNA expression profiles, which are obtained through a microarray experiment, are input (S710), the controller 140 may compute the correlation between a specific miRNA and a specific gene on the basis of the input experimental data (S720).

Afterwards, the computing apparatus 100 may extract a neighboring miRNA located within an effective distance from a specific miRNA input as experimental data by using a miRNA cluster database (S730). The miRNA cluster database includes distance data between miRNAs, and the computing apparatus 100 may determine that miRNA located within 10 kb (kilobase) from a specific miRNA is in an effective distance. However, the effective distance is not needed to be set to 10 kb, and may be varied in accordance with selection.

Afterwards, the computing apparatus 100 may compute a correlation value between a neighboring miRNA located to be adjacent to a specific miRNA within an effective distance and a gene (S740). For example, in the example shown in FIG. 6, if miRNA1 is a neighboring miRNA of miRNAi, the computing apparatus 100 may compute a correlation value for miRNA1-genem.

### Embodiment 3

### Computation of Correlation in Consideration of Transcription Factor

The computing apparatus 100 according to the present invention may compute a correlation value in consideration of a transcription factor between genes. Computation of the correlation value in consideration of a transcription factor between genes will be described with reference to the drawings which will be disclosed later.

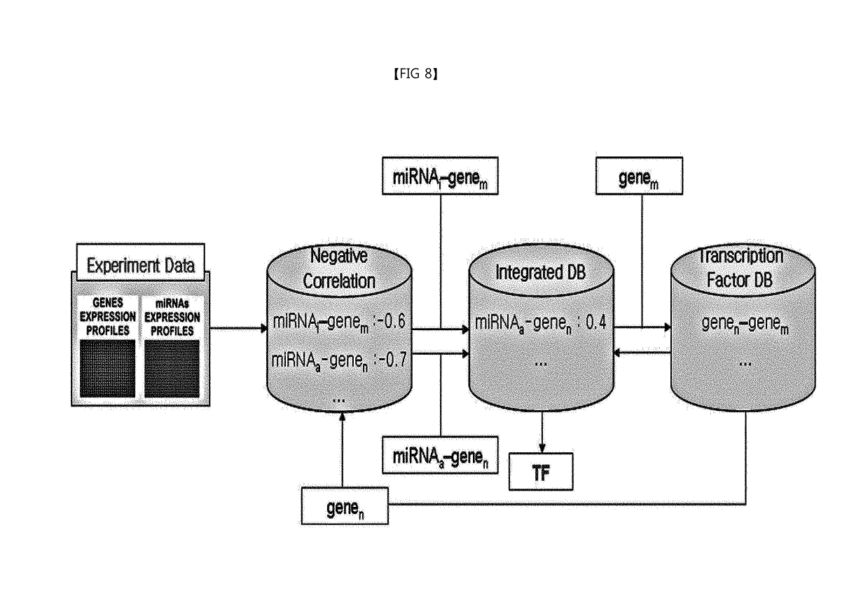

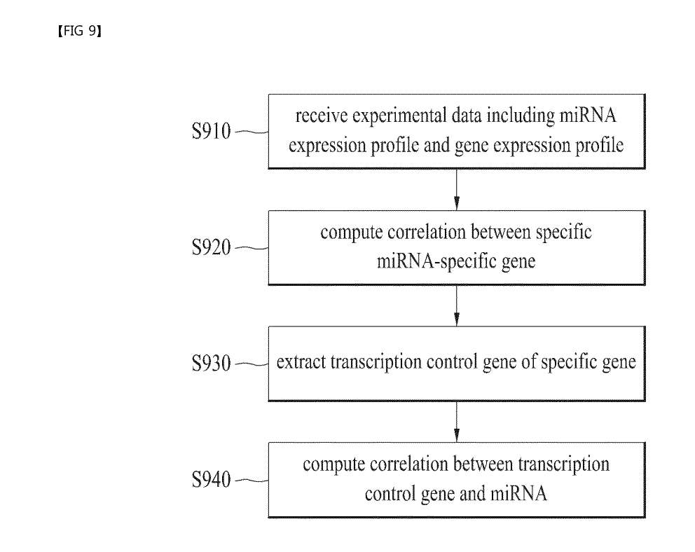

FIG. 8 is a conceptual diagram illustrating a method for computing a correlation value between a specific miRNA and a transcription control gene by using a transcription factor database, and FIG. 9 is a flow chart illustrating a method for computing a weight value between a specific miRNA and a transcription control gene by using a transcription factor database.

First of all, if experimental data that include genes expression profiles and miRNA expression profiles, which are obtained through a microarray experiment, are input (S910), the controller 140 may compute the correlation between a specific miRNA and a specific gene on the basis of the input experimental data (S920).

Afterwards, the computing apparatus 100 may identify existence or inexistence of a transcription control gene that activates or deactivates transcription of a specific gene by being specifically combined with a transcription control site DNA of the specific gene from a transcription factor database (S930).

If the transcription control gene of the specific gene exists, the computing apparatus 100 may compute a correlation value between the transcription control gene and miRNA (S940). For example, in the example shown in FIG. 8, if the transcription control gene of genem is genen, the computing apparatus 100 may compute the correlation value between miRNA-genem on the basis of the correlation value between miRNA-genen.

The computing apparatus 100 may compute an interaction score for a gene of a similar miRNA, an interaction score for a gene of a neighboring miRNA, and an interaction score for miRNA of the transcription control gene on the basis of the correlation values computed through the embodiments 1 to 3.

If the interaction score between miRNA-genes is obtained through a micro RNA target genes analysis algorithm, the computing apparatus 100 may extract a biomarker by using a list of specific expression genes of biliary tract cancer patients based on the differentially expression genes analysis algorithm.

A method for extracting a biomarker for a biliary tract cancer patient on the basis of the integrated analysis algorithm for extracting the aforementioned biomarker will be described in detail.

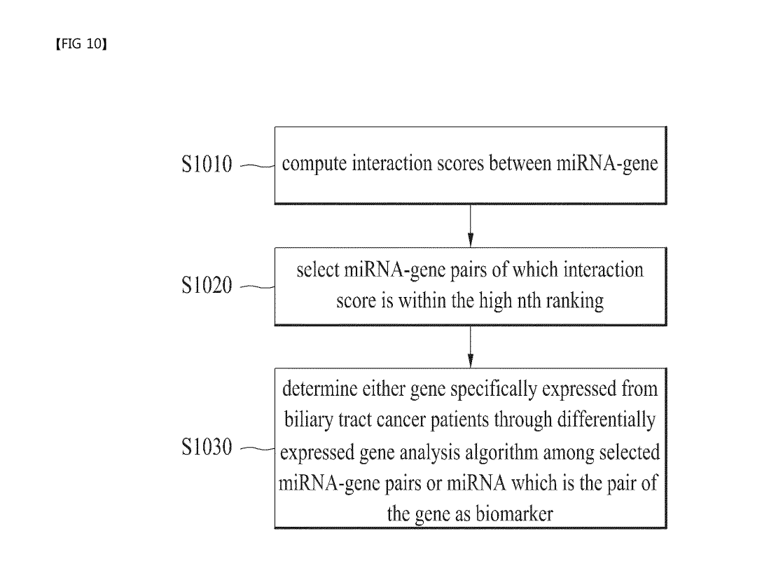

FIG. 10 is a flow chart illustrating a method for extracting a biomarker for a biliary tract cancer patient on the basis of an integrated analysis algorithm for extracting the biomarker. For convenience of description, it is assumed that the computing apparatus 100 stores a list of genes specifically expressed (for example, over-expressed or low-expressed) from biliary tract cancer patients unlike normal people by using the differentially expressed genes analysis algorithm.

Referring to FIG. 10, the computing apparatus 100 may compute the interaction score between miRNA-genes by using a micro RNA target gene analysis algorithm (S1010). Since the step of computing the interaction score is the same as that described with reference to FIGS. 4 to 9, its detailed description will be omitted.

Afterwards, the computing apparatus 100 may select a miRNA-gene pair of which interaction score is within the nth high ranking (S1020) and determine either intersection between genes in the selected miRNA-gene pair and a list of genes specifically expressed from biliary tract cancer patients unlike normal people on the basis of the differentially expressed genes analysis algorithm or a set of miRNAs which are the pair of genes which belong to the intersection as a biomarker for diagnosing biliary tract cancer (S1030). That is, the computing apparatus 100 may determine genes, which have high interaction scores and are specifically expressed from biliary tract cancer patients unlike normal people even in the differentially expressed genes algorithm, or a set of miRNAs which are the pair of the genes, as a biomarker for diagnosing biliary tract cancer.

For another example, the computing apparatus 100 may determine m number of genes in the order of high interaction scores of the miRNA-gene pair and determine intersection of a list of genes specifically expressed from biliary tract cancer patients unlike normal people on the basis of the differentially expressed genes analysis algorithm or miRNA which is the pair of genes belonging to the intersection, as a biomarker for diagnosing biliary tract cancer.

When six miRNA prediction tools of Targetscan, miRDB, DIANA-microT, PITA, miRanda, and MicroCosm are used and higher n number of genes (of which q-value is 0.05 or less and at the same time correlation value is −0.5 or less) among the interaction scores of the miRNA-gene pair are calculated, ACSM5, ADH6, ALDH1L1, APOA5, BHMT, CCL16, CYP1A2, CYP3A43, DAO, DDC, ESR1, F11, F13B, FETUB, GLYAT, GNMT, IGFALS, NAT2, PFKFB1, RDH16, SRD5A2, SULT2A1 and THRSP may be determined as biomarkers for diagnosing biliary tract cancer.

Individual features of the aforementioned biomarker are as follows.

ACSM5 (acyl-CoA synthetase medium-chain family member 5) has middle chain fatty acid:CoA ligase activity with wide substrate specificity. ACSM5 acts on acids which are C(4) to C(11) and corresponding 3-hydroxy-unsaturated acid and 2,3-unsaturated acid or 3,4-unsaturated acid.

ADH6 (alcohol dehydrogenase 6 (class V)) encodes class V alcohol dehydrogenase which is a member of an alcohol dehydrogenase family. The members of the alcohol dehydrogenase family metabolize various substrates including ethanol, retinol, other aliphatic alcohols, hydroxysteroid, and lipid peroxidation product. This gene is expressed even from a stomach as well as a liver, and contains a glucocorticoid response element above 5′ UTR which is a steroid hormone receptor combining site.

ALDH1L1 (aldehyde dehydrogenase 1 family, member L1) stimulates transformation of 10-formyltetrahydrofolate, nicotinamide adenine dinucleotide phosphate (NADP+) and H₂O to tetrahydrofolate, NADPH and CO₂. Loss of function or expression of this gene is associated with reduced apoptosis, increased cell migration, and tumor progression.

APOA5 (apolipoprotein A-V) is minor apolipoprotein, and may be associated with chylomicron. APOA5 is an important determination factor of a blood triglycerides (TG) level by means of a possible stimulator of apo-CII lipoprotein lipase (LPL) TG hydrolysis and an inhibitor of a liver VLDL-TG production rate (without an influence on a blood triglycerides production rate). APOA5 poorly activates lecithin:cholesterol acyltransferase (LCAT), and does not reinforce a leak of cholesterol from macrophage.

BHMT (betaine-homocysteine methyltransferase) is included in a control of homocysteine metabolism. Betaine and homocysteine are respectively transformed to dimethylglycine and methionine. This reaction is also required for irreversible oxidation of choline.

CCL16 (chemokine (C-C motif) ligand 16) represents chemotactic activity for lymphocyte and monocyte. CCL16 inhibits proliferation of a myeloid progenitor cell.

CYP1A2 (cytochrome P450, family 1, subfamily A, polypeptide 2), cytochrome P450 is a group of hemo-thiolate monooxygenase. In a liver microsome, this enzyme is involved in a NADPH-dependent electron transportation path and oxidizes various compounds which are not related with one another structurally and include steroid, fatty acid and xenobiotic. This enzyme has the best activity in stimulating 2-hydroxylation. Caffeine is mainly metabolized by cytochrome CYP1A2 in liver through initial N3-demethylation reaction. Also, this enzyme acts on metabolism of aflatoxin B1 and acetaminophen, and is involved in bio-activity of carcinogenic aromatic and heterocycle amine. This enzyme stimulates N-hydroxylation reaction of heterocycle amine and 0-deethylation reaction of phenacetin.

CYP3A43 (cytochrome P450, family 3, subfamily A, polypeptide 43) represents low testosterone 6-beta-hydroxylase activity.

DAO (D-amino-acid oxidase) controls a level of D-serine in a brain, wherein D-serine is a neuromodulator. DAO contributes to dopamine synthesis through high activity to D-DOPA. DAO may act as a detoxicator for detoxifying D-amino acid accumulated during aging. DAO acts on various D-amino acids with preference of D-amino acid that includes polar, aromatic and alkaline radicals subsequent to a small hydrophobic chain. DAO does not act on an acidic amino acid.

DDC (dopa decarboxylase (aromatic L-amino acid decarboxylase)) stimulates decarboxylation reactions of L-3,4-dehydroxyphenylalanine (DOPA) into dopamine, L-5-hydroxytryptophane into serotonin, and L-tryptophan into tryptamine.

ESR1 (estrogen receptor 1) is a nuclear hormone receptor. Steroid hormone and its receptor are involved in a control of eukaryotic cell gene expression, and have an effect on differentiation in cell proliferation and target tissue. A ligand-dependent nuclear transactivation is involved in direct homodimer binding with a palindromic estrogen response element (ERE) sequence or with other DNA-binding transcription factor such as AP-1/c-Jun, c-Fos, ATF-2, Sp1 and Sp3, thereby mediating ERE-independent signal transfer. Ligand binding derives morphologic change that allows sequence combination or binding combination with a multi-protein coactivator complex. Mutual transrepression occurs between an estrogen receptor (ER) and NF-kappa-B cell type-specifically. The ESR1 reduces NF-kappa-B DNA-binding activity, inhibits transcription NF-kappa-B-mediated from IL6 promotor, and shifts related coregulator and RELA/p65 from the promotor. The ESR1 may be gathered in NF-kappa-B reaction element of CCL2 and IL8 promotor, and may shift CREBBP. The ESR1 exists on ERE sequence together with NF-kappa-B component RELA/p65 and NFKB1/p50. Also, the ESR1 may activate transcription, which includes individual gathering of adjacent reaction elements, by arousing synergy with NF-kappa-B, and its function includes CREBBP. The ESR1 may stimulate transcription activity of TFF1. Also, the ESR1 may mediate membrane-initiated estrogen signal transfer that includes a kinase signal transfer cascade reaction. Isoform 3 is involved in activity of NOS3 and endothelial nitric oxide production. Isoform which is lack of one or several functional domains is regarded to control transcription activity by means of a claustrum receptor, competitive ligand or DNA binding and/or heterodimerization. Isoform 3 may be combined with ERE and may inhibit isoform 1.

F11 (coagulation factor XI) stimulates a middle phase of an intrinsic path of blood coagulation by activating a factor IX.

F13B (coagulation factor XIII, B polypeptide) has a chain B which is not catalytically active but stabilizes a subunit B and is regarded to control a transglutaminase formation rate by means of thrombin.

Protein encoded by FETUB (fetuin B) is a member of a fetuin family which is a part of a cystatin superfamily of a cysteine protease inhibitor. Fetuin is related to various functions including osteogenesis, bone resorption, a control of insulin and liver growth factor receptor, and a reaction to systemic inflammation.

GLYAT (glycine-N-acyltransferase) is a mitochondrial acyltransferase carrying an acyl group to N-terminus of glycine. Various N-acylglycines are formed by jointing a plurality of substrates, whereby xenobiotics such as benzoic acid or salicylic acid and endogenic organic acid such as isovaleric acid may be detoxified.

GNMT (glycine N-methyltransferase) forms N-methylglycine (sarcocine) together with subsidiary production of S-adenosylhomocysteine by stimulating methylation of glycine using S-adenosylmethionine (AdoMet). The GNMT may play an important role in a control of tissue concentration of AdoMet and a metabolic control of methionine.

IGFALS (insulin-like growth factor binding protein, acid labile subunit) is involved in a protein-protein interaction that causes protein complex, receptor-ligand binding or cell adhesion.

NAT2 (N-acetyltransferase 2 (arylamine N-acetyltransferase)) joins in detoxification during overdose of hydrazine and arylamine drugs. The NAT2 may stimulate N-acetylation or O-acetylation of various arylamine and heterocyclic amine substrates, or may biologically activate various carcinogens which are known.

PFKFB1 (6-phosphofructo-2-kinase/fructose-2,6-biphosphatase 1) synthesizes and decomposes fructose 2,6-bisphosphate.

RDH16 (retinol dehydrogenase 16 (all-trans)) is an oxidoreductase having preference to NAD. The RDH16 oxidizes retinol and 13-cys-retinol to aldehyde. The RDH16 has activity with respect to CRBP-binding retinol, which is higher than that with respect to free retinol. The RDH16 oxidizes 3-alpha-hydroxycysteroid. The RDH16 oxidizes androstenediol and androsterone to dihydrotestosterone and androstendione, and also stimulates a reverse reaction.

SRD5A2 (steroid-5-alpha-reductase, alpha polypeptide 2 (3-oxo-5 alpha-steroid delta 4-dehydrogenase alpha 2)) transforms testosterone (T) to 5-alpha-dehydrotestosterone (DHT), and transforms progesterone or corticosterone to corresponding 5-alpha-3-oxosteroid. The SRD5A2 plays an important role in sexual differentiation and androgen physiology.

SULT2A1 (sulfotransferase family, cytosolic, 2A, dehydroepiandrosterone (DHEA)-preferring, member 1) is a sulfotransferase that stimulates sulfonization of steroid and bile in liver and adrenal gland by using 3′-phospho-5′-adenylsulfate (PAPS) as a sulfate donor.

THRSP (thyroid hormone responsive (SPOT14 homolog, rat)) serves to control lipid synthesis in lactation mammary gland. The THRSP is important in bio-synthesis of triglyceride having a middle length fatty acid chain. The THRSP may control lipid synthesis by interacting with MID1IP1 and preventing interaction with ACACA. The THRSP may serve as a coactivator, and may control transcription factor activity of THRB.

Meanwhile, when six miRNA prediction tools of Targetscan, miRDB, DIANA-microT, PITA, miRanda, and MicroCosm are used and tissue is used as a biological sample, as miRNA set which corresponds to a pair of higher n number of genes (of which q-value is 0.05 or less and at the same time correlation value is −0.5 or less) among the interaction scores of the miRNA-gene pair, hsa-miR-21-5p, hsa-miR-93-5p, hsa-miR-106b-5p, hsa-miR-155-5p, hsa-miR-181a-5p, hsa-miR-200a-3p, hsa-miR-200b-3p, hsa-miR-222-3p, and hsa-miR-331-3p may be determined as biomarkers for diagnosing biliary tract cancer.

Also, when blood is used as a biological sample, hsa-miR-21-5p, hsa-miR-93-5p, hsa-miR-106b-5p, hsa-miR-155-5p, hsa-miR-181a-5p, and hsa-miR-222-3p are used as biomarkers for diagnosing biliary tract cancer.

Base sequence of each miRNA which belongs to the aforementioned biomarkers is as illustrated in Table 2 below.

TABLE 2

| Mature_id | miRNA_id | Sequence |
|---|---|---|
| hsa-miR-21-5p | hsa-mir-21 | UAGCUUAUCAGACUGAUG |
| | | UUGA |
| hsa-miR-93-5p | hsa-mir-93 | CAAAGUGCUGUUCGUGCA |
| | | GGUAG |
| hsa-miR-106b-5p | hsa-mir-106b | UAAAGUGCUGACAGUGCA |
| | | GAU |
| hsa-miR-155-5p | hsa-mir-155 | UUAAUGCUAAUCGUGAUA |
| | | GGGGU |
| hsa-miR-181a-5p | hsa-mir-181a-1, | AACAUUCAACGCUGUCGG |
| | hsa-mir-181a-2 | UGAGU |
| hsa-miR-200a-3p | hsa-mir-200a | UAACACUGUCUGGUAACG |
| | | AUGU |
| hsa-miR-200b-3p | hsa-mir-200b | UAAUACUGCCUGGUAAUG |
| | | AUGA |
| hsa-miR-222-3p | hsa-mir-222 | AGCUACAUCUGGCUACUG |
| | | GGU |
| hsa-miR-331-3p | hsa-mir-331 | GCCCCUGGGCCUAUCCUA |
| | | GAA |

Experimental procedure and result of the biomarkers for diagnosing biliary tract cancer, which are obtained by the result, will be described in detail.

Biliary Tract Cancer Patient Sample and Microarray Experiment

A Liver Cancer Institute and Zhongshan Hospital (Fudan university in shanghai, China) in 2002 and 2003 and Kanazawa University Hospital (ishikawa, Japan) in 2008 to 2010 have obtained tissues of intrahepatic cholangiocellular carcinoma (ICC) and combined hepatocellular cholangiocarcinoma (CHC) from Asian patients subjected to curative resection through prior consent.

Sample has been approved by an Institutional Review Board, and has been written by National Institutes of Health (NIH) of Human Subjects Research in USA. A total of 23 ICC and CHC cases have been used to make mRNA and microRNA signatures. Biliary tract cancer has been early diagnosed on the basis of serum test and imaging, and early diagnosis of biliary tract cancer has been identified histopathologically by pathologists. Characterization of 68 Caucasian ICC patients from an independent group has been disclosed (Hepatology, Vol. 56, No. 5, 1792-803).

Verification of Biomarker Set of the Present Invention

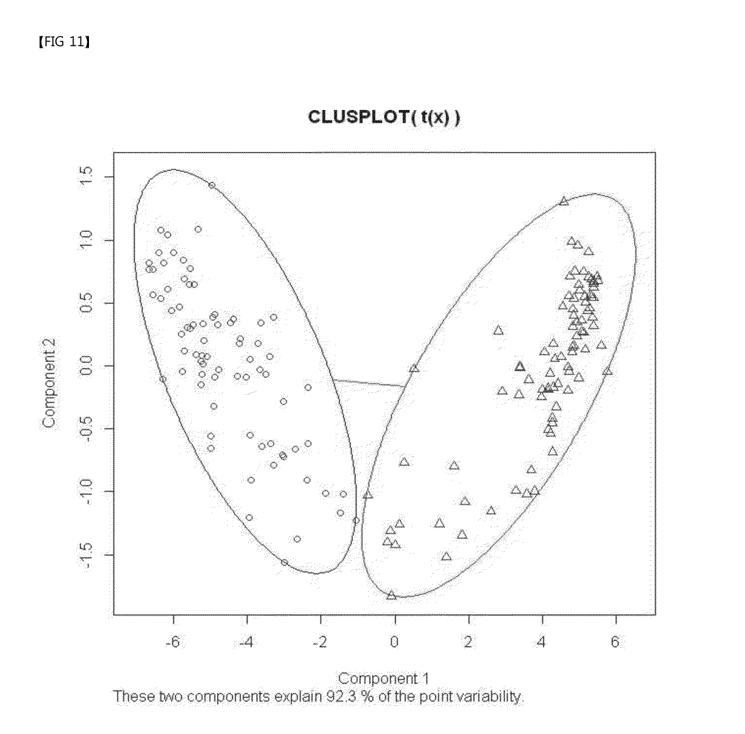

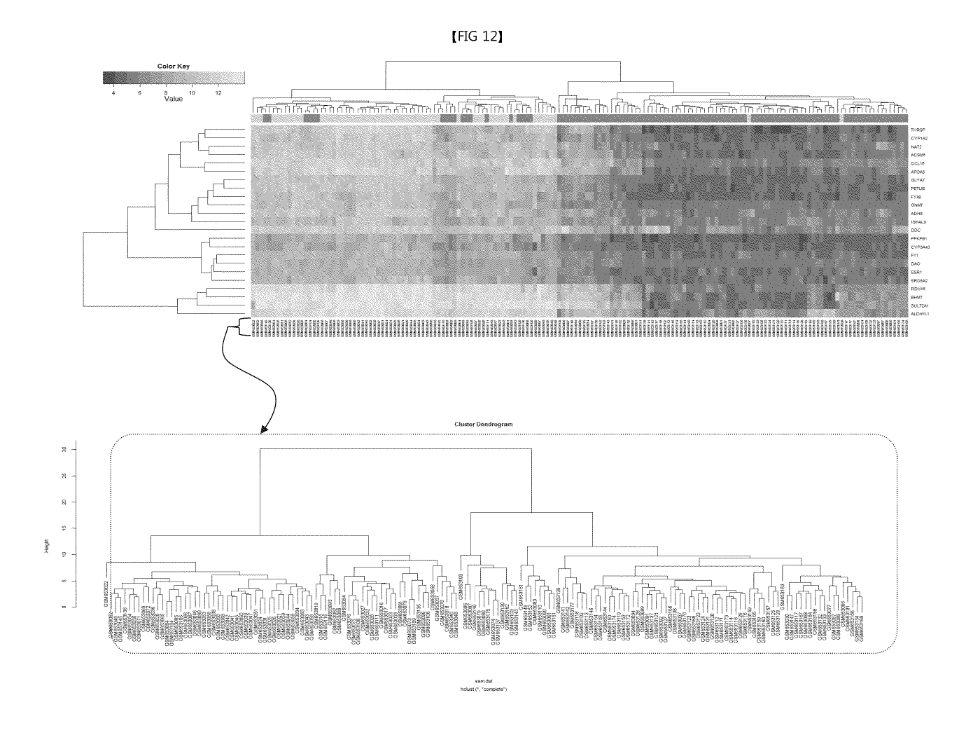

In the present invention, verification in diagnosis of biliary tract cancer of a gene biomarker set has targeted a total of 163 people including 104 biliary tract cancer patients and 59 normal people. Biliary tract cancer has been diagnosed by principal component analysis and a hierarchical clustering, euclidean distance, complete method by using GEO (Gene Expression Omnibus) data GSE26566 through blood gathered from the targets. As a result, sensitivity to biliary tract cancer was 82% ( 85/104) and specificity was 97% ( 57/59). FIG. 11 illustrates a plot of an analysis result of principal components. In FIG. 11, component 1 of a horizontal axis may mean a first principal component PC1, and component 2 of a vertical axis may mean a second principal component PC 2. Moreover, entities expressed by a triangle may mean cancer patients, and objects expressed by a circle may mean normal people. FIG. 12 illustrates a heat map of a hierarchical cluster analysis result. In FIG. 12, red bars located on a top of the heat map may mean cancer patients, and blue bars may mean normal people.

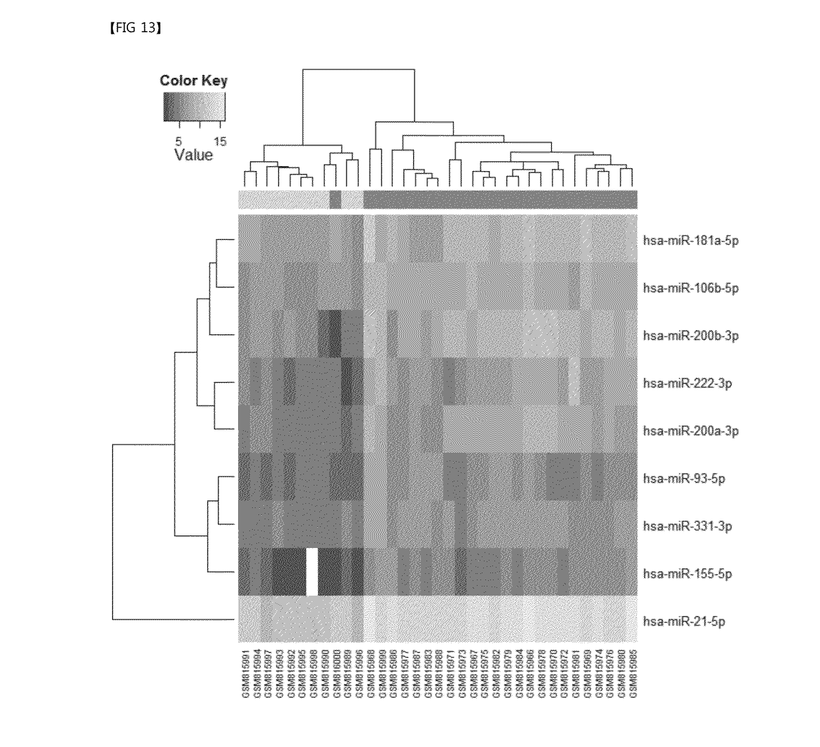

Meanwhile, in the present invention, verification in diagnosis of biliary tract cancer of a microRNA biomarker set for a tissue sample has targeted a total of 35 people including 25 biliary tract cancer patients and 10 normal people. Biliary tract cancer has been diagnosed by a hierarchical clustering, euclidean distance, complete method by using GEO (Gene Expression Omnibus) data GSE32957 through blood gathered from the targets. As a result, sensitivity to biliary tract cancer was 96% ( 24/25) and specificity was 100% (10/10), which correspond to a very excellent range. FIG. 13 illustrates a heat map of a hierarchical cluster analysis result based on data GSE32957. In FIG. 13, red bars located on a top of the heat map mean cancer patients, and blue bars mean normal people.

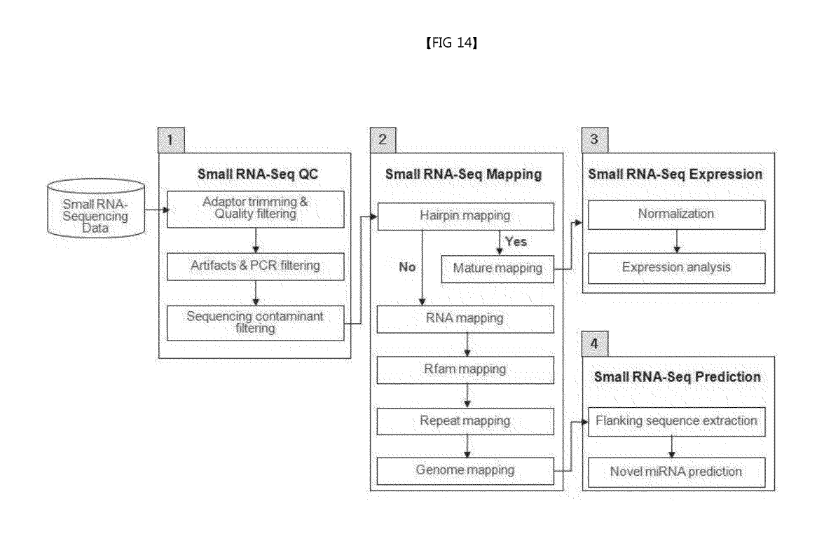

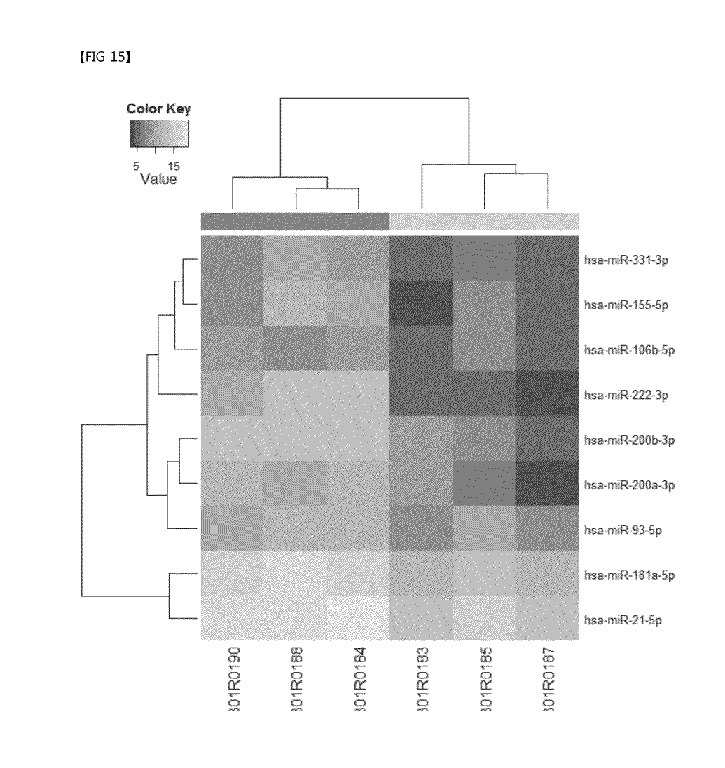

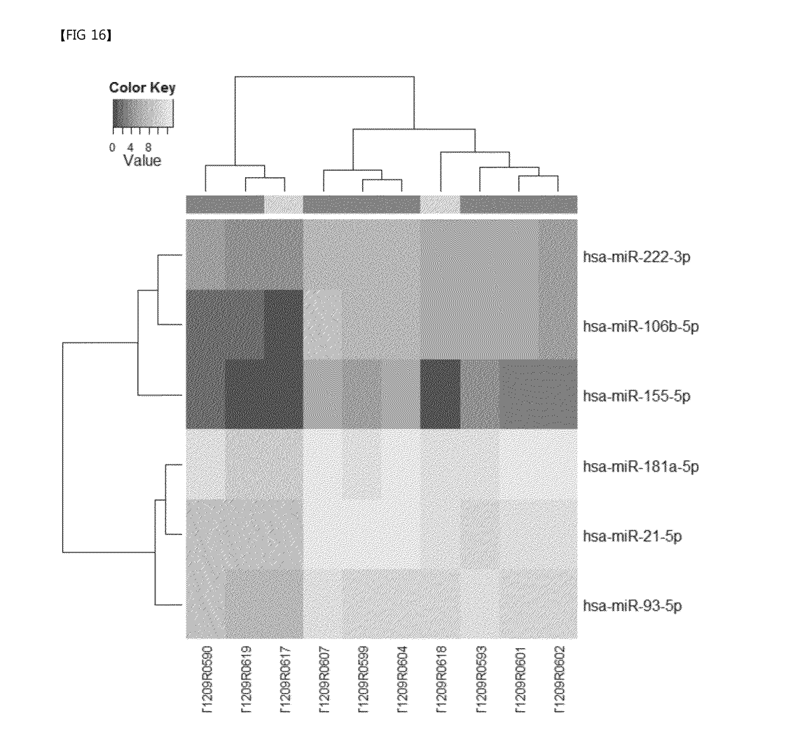

Also, verification in diagnosing biliary tract cancer of microRNA biomarkers for tissue sample and blood sample in accordance with the present invention has been performed separately. First of all, the biomarker for tissue sample has targeted a total of 4 people including 2 biliary tract cancer patients and 2 normal people, and the biomarker for blood sample has targeted 8 biliary tract cancer patients and 2 normal people. The biliary tract caner has been diagnosed by a hierarchical clustering, euclidean distance, complete method based on small RNA sequencing data which is a Next Generation Sequencing (NGS) method by using tissue and blood gathered from the targets. General description of the small RNA sequencing data analysis is disclosed in FIG. 14. As a result, sensitivity to biliary tract cancer of the biomarker for tissue sample was 100% (3/3) and specificity was 100% (3/3). In this case, when small RNA sequencing data are used, the result of hierarchical clustering analysis is shown in FIG. 15. Also, sensitivity to biliary tract cancer of the biomarker for blood sample was 75% ( 6/8) and specificity was 50% (½). In this case, when small RNA sequencing data are used, the result of hierarchical clustering analysis is shown in FIG. 16. In FIGS. 15 and 16, red bars located on a top of the heat map mean cancer patients, and blue bars mean normal people.

Meanwhile, the aforementioned biomarker is used as an apparatus for diagnosing biliary tract cancer. Examples of the apparatus for diagnosing biliary tract cancer include a diagnosis chip, a diagnosis kit, a quantitative PCR (qPCR) equipment, a POCT equipment, and a sequencer. In the diagnosis chip, the diagnosis kit, the quantitative PCR (qPCR) equipment, the POCT equipment and the sequencer, known parts may be used as parts except the biomarker set.

According to one embodiment of the present invention, the aforementioned methods may be implemented in a medium in which programs are recorded, as a code that can be read by a processor. Examples of the medium that may be read by the processor include a ROM, a RAM, CD-ROM, a magnetic tape, a floppy disk, and an optical data memory. Also, another example of the medium may be implemented in a type of carrier wave (for example, transmission through Internet). The configuration and method of the aforementioned embodiments are not limited to the computing apparatus 100 described as above, and the computing apparatus 100 may be configured by selective combination of all or some of the embodiments such that various modifications may be made in the embodiments.

### INDUSTRIAL APPLICABILITY

Although various embodiments of the present invention described as above have been described based on the method for extracting a biomarker for diagnosing biliary tract cancer, the embodiments of the present invention may equally be applied to various methods for extracting biomarkers for diagnosing various cancers.

1. A method for extracting a biomarker for diagnosing biliary tract cancer, the method comprising the steps of:
computing interaction scores obtained by digitalizing complementary combination levels between microRNA and gene;
determining n number of microRNA and gene pairs having high interaction scores; and
extracting either a gene common to a gene specifically expressed from biliary tract cancer patients among the n number of microRNA and gene pairs or microRNA which is the pair of the gene.

2. The method according to claim 1, wherein the step of computing the interaction scores includes:
acquiring at least one database obtained by statistically processing prediction scores between microRNAs and genes;
computing a normalized score from the prediction scores between microRNAs and genes;
computing a binding ranking of microRNA per gene and a binding ranking of gene per microRNA on the basis of the normalized score; and
computing the interaction scores on the basis of the binding ranking of microRNA and the binding ranking of gene.

3. The method according to claim 2, wherein the at least one database is generated using a microRNA target prediction tool.

4. The method according to claim 3, wherein the microRNA target prediction tool includes at least one of Targetscan, miRDB, DIANA-microT, PITA, miRanda MicroCosm, RNAhybrid, PicTar and RNA22.

5. The method according to claim 2, wherein the normalized score is computed on the basis of a ranking of prediction scores of microRNA and gene pairs in the at least one database.

6. The method according to claim 5, wherein the normalized score is computed in accordance with Equation 1 below:
$${\sum }_{i = 1}^{n} \frac{( {T}_{i} + 1 - {R}_{i , j} )}{{T}_{i}} \qquad [ Equation 1 ]$$
(in the Equation 1, i means the ith database, n means the number of databases, Ti may mean a total number of microRNA-gene pairs in the ith database, and Ri,j means a ranking of the jth microRNA-gene pair in the ith database).

7. The method according to claim 5, wherein the interaction scores are computed on the basis of a ranking of microRNA per gene and a ranking of gene per microRNA on the basis of the normalized score.

8. The method according to claim 7, wherein the interaction scores are computed in accordance with Equation 2 below:
$$( \frac{{t}_{m i} + 1 - {r}_{m i}}{{t}_{m i}} ) \times ( \frac{{t}_{gj} + 1 - {r}_{gj}}{{t}_{gj}} ) \qquad [ Equation 2 ]$$
(in the Equation 2, tmi means the number of ith miRNAi-gene pairs, tgj means the number of genej-miRNA pairs, rmi means a ranking of the normalized score of the ith miRNA for the jth gene, and rgj means a ranking of the normalized score of the jth gene for the ith microRNA).

9. A computing apparatus comprising:
a memory for storing data; and
a controller for computation,
wherein the controller is configured to compute interaction scores obtained by digitalizing complementary combination levels between microRNA and gene, determine n number of microRNA and gene pairs having high interaction scores, and extract either a gene common to a gene specifically expressed from biliary tract cancer patients among the n number of microRNA and gene pairs or microRNA which is the pair of the gene.

10. A biomarker for diagnosing biliary tract cancer, comprising ACSM5, ADH6, ALDH1L1, APOA5, BHMT, CCL16, CYP1A2, CYP3A43, DAO, DDC, ESR1, F11, F13B, FETUB, GLYAT, GNMT, IGFALS, NAT2, PFKFB1, RDH16, SRD5A2, SULT2A1 and THRSP.

11. A biomarker for diagnosing biliary tract cancer, comprising hsa-miR-21-5p, hsa-miR-93-5p, hsa-miR-106b-5p, hsa-miR-155-5p, hsa-miR-181a-5p, hsa-miR-200a-3p, hsa-miR-200b-3p, hsa-miR-222-3p, and hsa-miR-331-3p, which use tissue as a biological sample.

12. A biomarker for diagnosing biliary tract cancer, comprising hsa-miR-21-5p, hsa-miR-93-5p, hsa-miR-106b-5p, hsa-miR-155-5p, hsa-miR-181a-5p, and hsa-miR-222-3p, which use blood as a biological sample.

13. An apparatus for diagnosing biliary tract cancer, comprising the biomarker of claim 10.

14. The apparatus according to claim 13, wherein the apparatus is any one of a diagnosis chip, a diagnosis kit, a quantitative PCR (qPCR) equipment, a POCT equipment, and a sequencer.
